(19) United States
(12) Patent Application Publication
(10) Pub. No.: US 2009/0093836 A1
(43) Pub. Date: Apr. 9, 2009

(54) Methods for reducing hydrostatic organ pressure

(76) Inventor: Yair Feld, Haifa (IL)

Correspondence Address: MARTIN D. MOYNIHAN d/b/a PRTSI, INC., P.O. BOX 16446, ARLINGTON, VA, 22215, US

(21) Appl. No.: 11/919,907

(22) PCT Filed: May 4, 2006
(86) PCT No.: PCT/IL2006/000525

§ 371 Date: Nov. 5, 2007

Related U.S. Application Data
(60) Provisional application No. 60/594,772, filed on May 5, 2005.
(60) Provisional application No. 60/678,817, filed on May 9, 2005.

(51) Int. Cl. A61M 29/00; A61B 17/00

(52) U.S. Cl. 606/191; 128/898

(57) ABSTRACT

A method for stretching at least a portion of an organ to decrease interstitial hydrostatic pressure and improve at least one organ function. The method comprises providing at least one elastically compressible anchor, compressing the at least one anchor, anchoring the at least one anchor to a portion of an organ from the group of organs consisting of: a kidney, a liver, a bladder, and a stomach. The method further comprises releasing the compressing, thereby stretching the portion and decreasing interstitial hydrostatic pressure.

### FIELD OF THE INVENTION

The present invention relates generally to methods and devices for changing the hydrostatic pressure in an organ from the group of organs consisting of: kidney, bladder, stomach, and liver; thereby improving at least one aspect of organ function.

### BACKGROUND OF THE INVENTION

### Chronic Renal Failure

Chronic renal failure (CRF) is a progressive disease characterized by an increasing inability of the kidney to maintain normal levels of protein metabolism (such as urea), normal blood pressure, hematocrit, sodium, water, potassium, and acid-base balance. Once the process of nephron destruction begins, it appears that there is a compensatory hyperfiltration in other nephrons. This in turn likely leads to sclerosis, and tubular hypertrophy in the compensating nephrons, resulting in increased expenditure of energy, greater consumption of oxygen, and production of reactive oxygen metabolites, and tubulointerstitial damage. (Cecil Essential of Medicine, Andreoli)

When CRF reduction dips to 90%, and/or serum creatinine in an adult reaches about 3 mg/dL, and no factors in the renal disease are reversible, the renal disease is highly likely to progress to end-stage renal disease (ESRD) over a variable period (from a few years to as many as 20 to 25 years). There are 4.5 million patients with ESRD in the USA, and about 100,000 new cases every year. The number of ESRD patients increases annually at a rate of about 5-8% due to aging and type II DM. (Lancet 2005; 365: 331-40)

Major causes of CRF include type II DM and hypertension. Management of CRF may include treatment, primarily through diet, of underlying cause, whether type II DM or hypertension.

Following kidney failure, the options for treatment include dialysis, and kidney transplantation. (Cecil Essentials of Medicine, Andreoli)

Dialysis can be performed on acute or chronic renal failure patients. Despite improved technologies, the annual mortality rates in the United States still average around 20% per year (Cecil Textbook of Medicine, 21^{st} edition. W.B. Saunders Company, 2000).

### Obesity

Obesity is a major cause of morbidity, and mortality. Food intake is a system that is regulated by a variety of nerves providing signals to the central nervous system (CNS).

Afferent signals provide information to the CNS, which is the centre for the control of satiety or food seeking. A subset of vagal afferents that have been implicated as key to gastrointestinal (GI) regulation, and ingestive behavior consists of the two morphologically distinct classes of mechanoreceptors supplied to the muscle wall of the GI tract.

One class is comprised of intraganglionic laminar endings (IGLEs) that innervate myenteric ganglia, and are distributed throughout the GI tract. The other consists of intramuscular arrays (IMAs) that have a much more restricted distribution that is limited to the stomach, and adjacent sphincters.

In particular, IMAs are concentrated in the circular or longitudinal muscle layers of the fore stomach, and lower esophageal, and pyloric sphincters, where they form appositions with muscle fibers, and/or interstitial cells of Cajal.

These vagal mechanoreceptors are thought to provide the CNS with negative feedback that is activated by accumulation, and movement of food in the stomach and intestines, and may therefore be involved in regulation of feeding, especially in the control of meal size or short-term satiety (Fox E A et al. J Neurosci. 1 Nov. 2001; 21(21):8602-15.).

### Urinary Incontinence

Urinary incontinence is a major social, and hygiene problem. Urinary incontinence is generally classified into three types: stress incontinence, urge incontinence, and mixed incontinence. Detrusor instability (urge incontinence) is characterized by spontaneous and uninhibited contraction of the detrusor muscle during bladder filling. The bladder pressure exceeds the urethral pressure resulting in incontinence. Treatment of detrusor instability is based on inhibiting the symptoms of urgency, and increasing the interval between voids. Options include bladder training, biofeedback, hypnosis, and drugs. Surgery may also be considered either to interrupt the nervous pathways or to increase bladder capacity. A common approach is resection of the vesicle plexus approached vaginally.

### Cirrhosis

Cirrhosis, loss of liver function, affects the body in many ways. For example, when the liver loses its ability to make the protein albumin, water accumulates in the legs (edema) and abdomen (ascites). Additionally, a damaged liver cannot remove toxins from the bloodstream, causing them to accumulate in the blood and eventually the brain. There, toxins can dull mental functioning and cause personality changes, coma, and even death. Signs of the buildup of toxins in the brain include neglect of personal appearance, unresponsiveness, forgetfulness, trouble concentrating, or changes in sleep habits.

### PRIOR ART

Stomach restriction devices and methods are known.

Gastric anchors are known. For example Imran teaches a variety of anchor designs, and methods of configuration, in multiple patents, including: U.S. patent application Ser. No. 11/992,382, published on 30 Jun. 2005 as U.S. 2005/0143784 A1, which teaches devices and methods to anchor sensors to a gastric portion; and U.S. patent application Ser. No. 10/991,648 published on 20 Jun. 2005 as U.S. 2005/0143760 A1, which teaches devices and methods to interconnect anchors to gastric tissue, and limit gastric expansion.

The contents of all of the above-noted applications are hereby incorporated by reference as if fully set forth herein.

### SUMMARY OF THE INVENTION

Embodiments of the present invention successfully address at least some of the shortcomings of the prior art by providing methods, and devices for reducing hydrostatic pressure in at least one portion of an organ from the group of organs consisting of a kidney, bladder, stomach, and liver, thereby improving at least one aspect of organ function.

According to the teachings of the present invention, there is provided a method for stretching at least a portion of an organ. The method comprises, providing at least one elastically compressible anchor, compressing the at least one anchor, anchoring the at least one anchor to a portion of an organ from the group of organs consisting of a kidney, a bladder, a liver, and a stomach, and releasing the compressing, thereby stretching the portion of the organ.

According to the teachings of the present invention, there is provided a method for stretching at least a portion of an organ. The method comprises, stretching a portion of an organ from the group of organs consisting of a kidney, a liver, a bladder, and a stomach, and anchoring at least one anchor to the portion, thereby at least partially maintaining the stretching.

In some embodiments, at least a portion of at least one anchor is oriented at an angle relative to an external surface of the portion of the organ at an angle of between 0 degrees and 20 degrees from parallel to the surface.

In some embodiments, at least a portion of at least one anchor is oriented at an angle relative to an external surface of the portion of the organ, at an angle of between 20 degrees, and 70 degrees from parallel to the surface.

In some embodiments, at least a portion of at least one anchor is oriented at an angle relative to an external surface of the portion of the organ, at an angle of between about 70 degrees and about 90 degrees from parallel to the surface.

In some embodiments, the at least one anchor comprises at least two magnets, each magnet having a magnetic field, wherein the compressing includes bringing same polarity of the magnetic fields toward each other so as to increase the magnitude of a repulsive magnetic force produced by the magnetic fields of the magnets.

In some embodiments, the at least one anchor comprises at least two magnets, each magnet having a magnetic field, wherein, the anchoring includes anchoring the at least two magnets at a distance, and an orientation so that respective the magnetic fields apply a force to substantially maintain the stretching.

In some embodiments, an axis passing through the at least two magnets is oriented at an angle relative to an external surface of the portion at an angle of between about 0 degrees and about 20 degrees from parallel to the surface.

In some embodiments, an axis passing through the at least two magnets is oriented at an angle relative to an external surface of the portion, at an angle of between about 20 degrees and about 70 degrees from parallel to the surface.

In some embodiments, an axis passing through the at least two magnets is oriented at an angle relative to an external surface of the portion, at an angle of between about 70 degrees and about 90 degrees from parallel to the surface.

According to the teachings of the present invention, there is also provided a method for stretching at least a portion of an organ, the method comprises stretching a portion of an organ portion from the group of organs consisting of a kidney, a bladder, a liver, and a stomach, connecting a first end of a connector to the organ, the at least one connector having a body, a first end, and a second end, connecting the at least one connector second end to the substantially external portion at a distance from the first end, releasing the stretching, so that the stretching is at least partially maintained by the at least one connector.

According to the teachings of the present invention, there is also provided a method for stretching at least a portion of an organ. The method comprises, compressing at least one elastically compressible connector, the connector having a body, a first end, and a second end, connecting the at least one connector first end to an organ from the group of organs consisting of a liver, a kidney, a bladder, and a stomach, connecting the at least one connector second end to the organ portion at a distance from the first end, and releasing the compressing, thereby stretching the portion.

In some embodiments, the at least one connector is shaped so as to substantially follow a contour of at least part of an external boundary of the external organ portion.

In some embodiments, the at least one connector is elastically compressible along a longitudinal axis running through the body, the first end, and the second end. Further, the compressing comprises compressing the at least one connector first end toward the second end, along the longitudinal axis.

In some embodiments, the connector is substantially a longitudinally compressible spring, such as a helical spring.

In some embodiments, the at least one connector body is elastically deformable, and the compressing comprises bringing the first connector end toward the second connector end, thereby elastically deforming the body while displacing the body a distance from an axis running through the first, and second ends.

In some embodiments, the connector is substantially a leaf spring.

In some embodiments, the connecting comprises, providing at least two anchors, each anchor having a first end, a second end, and a body, anchoring the first end of the anchors to the organ portion so that the second end of the anchors protrudes from the organ, coupling the first end of the connector, and the second end of the connector each to the protruding second end of the anchor.

In some embodiments, the connecting comprises applying an adhesive between the first, and second ends of the at least one connector, and the organ portion.

In some embodiments, the adhesive comprises carboxymethyl cellulose.

In some embodiments, the connecting additionally comprises applying the adhesive to the connector body between the connector, and the portion.

In some embodiments, the connecting comprises applying the adhesive substantially to the entire contact area between the connector body, and the organ portion.

According to the teachings of the present invention, there is also provided a method for stretching at least a portion of an external boundary of an organ substantially outward. The method comprises, outwardly stretching a portion of a substantially external boundary of an organ, the organ selected from the group of organs consisting of a kidney, a liver, a bladder, and a stomach, and, conjoining the portion to at least one offset so that the stretching is at least partially maintained.

In some embodiments, the at least one offset location comprises a bone from the group consisting of ribs and vertebrae.

In some embodiments, the conjoining comprises anchoring the portion of a substantially external boundary of the organ to a first end of at least one anchor having a first end, and a second end, and attaching the anchor second end to the bone.

In some embodiments, the conjoining comprises suturing the portion of a substantially external boundary of the organ to the bone using at least one suture.

In some embodiments, the at least one offset comprises a curved body portion of at least one connector having a first end, a second end, and a body wherein the first end, and the second end are connected proximate to the portion of a substantially external boundary of the organ.

In some embodiments, the connecting comprises applying an adhesive between the connector body portion, and an outer surface of the organ.

In some embodiments, the adhesive comprises carboxymethyl cellulose.

In some embodiments, the adhesive is additionally applied to the first and the second ends of the at least one connector.

In some embodiments, the adhesive is applied on the entire contact area between the connector and the organ.

In some embodiments, the connecting comprises anchoring the portion of a substantially external boundary of the organ to a first end of at least one anchor having a first end, and a second end, and coupling the second end of the anchor to the connector body.

In some embodiments, the first end and the second end of the at least one connector are coupled to a first and a second anchor respectively, and the connecting comprises anchoring the first, and second anchors to the organ proximate to the portion of a substantially external boundary of the organ.

According to the teachings of the present invention, there is also provided a method for stretching at least a portion of an external boundary of an organ substantially outward. The method comprises, stretching at least one elastically stretchable anchor, the at least one anchor comprising a first end, and a second end, anchoring the first end of the at least one anchor to an organ, the organ selected from the group of organs consisting of a kidney, a liver, a bladder, and a stomach, and conjoining the second end of the at least one anchor with at least one offset so that the stretching is at least partially maintained.

In some embodiments, the at least one offset comprises a bone from the group consisting of ribs and vertebrae.

In some embodiments, the at least one offset comprises a curved body portion of at least one connector having a first end, a second end, and the body, wherein the first end, and the second end are implanted proximate to the portion of a substantially external boundary of the organ.

In some embodiments, the at least one offset comprises a body of at least one connector having a first end, a second end, and a body, wherein the first end, and the second end are attached to a first anchor, and a second anchor respectively, and the connecting comprises connecting the first, and second anchors proximate to the portion of a substantially external boundary of the organ.

In some embodiments, the stretching includes a linear component substantially parallel to an external boundary of the organ.

In some embodiments, the stretching is at least about 0.5 centimeters. Alternatively, the stretching is at least about 1.0, 1.5, 2.0, or 2.5 centimeters.

In some embodiments, the stretching is no more than about 5.0 centimeters. Alternatively, the stretching is no more that about 4.5, 4.0, 3.5, or 3.0 centimeters.

In some embodiments, the stretching includes an outward component, substantially perpendicular to an external boundary of the organ.

In some embodiments, the stretching is at least 0.5 centimeters Alternatively, the stretching is at least about 1.0, 1.5, 2.0, or 2.5 centimeters.

In some embodiments, the stretching is no more than about 5.0 centimeters. Alternatively, the stretching is no more that about 4.5, 4.0, 3.5, or 3.0 centimeters.

In some embodiments, the at least two anchors comprise at least three anchors.

In some embodiments, the at least one connector comprises at least two connectors.

In some embodiments, the at least two connectors substantially describe a line.

In some embodiments, the at least two connectors substantially describe an open polygon.

In some embodiments, the at least two anchors comprise at least four anchors.

In some embodiments, the at least one connector comprises at least three connectors.

In some embodiments, the at least three connectors substantially describe a line.

In some embodiments, the at least three connectors substantially describe a closed polygon.

In some embodiments the at least three connectors substantially describe an open polygon.

In some embodiments, the at least one connector comprises a sheet material.

In some embodiments, the at least two connectors comprise a substantially continuous single connector, comprising a sheet material.

In some embodiments, the at least three connectors comprise a substantially continuous single connector, comprising a sheet material.

In some embodiments the sheet material is selected from the group consisting of meshes, and nets.

In some embodiments, the material includes openings having an area of at least about 0.5 mm2. Alternatively, the openings have an area of at least about 0.75, 1.0, 1.25, 1.50, or 2.0 mm2.

In some embodiments, the material includes openings having an area of no more than about 2.0 mm2. Alternatively, the openings have an area of no more that about 0.50, 0.75, 1.0, 1.25, or 1.50 mm2.

In some embodiments, at least a portion of at least one anchor comprises at least one screw thread.

In some embodiments, the implanting includes screwing the at least one screw thread into the portion.

In some embodiments the organ comprises a kidney.

In some embodiments, the portion comprises a cortex of a kidney.

In some embodiments, the at least one stretched portion comprises a tissue located substantially in a kidney cortex.

In some embodiments, the portion comprises a tissue from the group consisting of a kidney cortex, medulla, ureter, and pelvis.

In some embodiments the stretched portions comprise a tissue from the group consisting of a kidney cortex, medulla, and pelvis.

In some embodiments, the stretched portions include at least one Bowman's capsule.

In some embodiments, the stretched portions include at least one renal corpuscle.

In some embodiments, the stretched portions include at least one loop of Henle.

In some embodiments, the stretched portions include at least one collecting duct.

In some embodiments, the stretching reduces pressure in at least one kidney filtration structure.

In some embodiments, the reduction of pressure occurs in a renal structure from the group of renal structures comprising a Bowman's capsule, a renal corpuscle, a loop of Henle, a collecting duct, and a convoluted tube.

In some embodiments the stretching leads to an increased glomerular filtration rate.

In some embodiments, the stretching leads to an increased osmotic pressure.

In some embodiments, the increased osmotic pressure occurs in at tissue from the group consisting of a loop of Henle, a renal corpuscle, a glomerulus, and a Bowman's capsule.

In some embodiments, the organ comprises a liver.

In some embodiments, the implanting causes increased blood flow through at least a portion of the liver.

In some embodiments, the implanting improves at least one liver homeostatic function with respect to the group of homeostatic compounds consisting of glucose, proteins, fat, cholesterol, hormones, and vitamins.

In some embodiments, the at least one liver homeostatic function, comprises homeostasis of a vitamin from the group consisting of vitamins A, D, E, and K.

In some embodiments, the implanting causes improved liver synthesis of at least one compound from the group consisting of proteins, bile acids, and cholesterol.

In some embodiments, the improved liver synthesis results in improved synthesis of at least one clotting factor.

In some embodiments, the implanting improves liver storage of at least one compound from the group consisting of vitamins, and cholesterol.

In some embodiments, the implanting improves liver excretion of at least one compound from the group consisting of cholesterol, bile acids, phospholipids, bilirubin, drugs, and poisons.

In some embodiments, the implanting improves liver filtration of at least one compound from the group consisting of gut poisons, nutrients, sugar, fat, bilirubin, bile acids, and immunoglobulins.

In some embodiments, the improved filtration of nutrients includes filtration of at least one amino acid.

In some embodiments, the improved filtration of immunoglobulins includes filtration of at least IgA.

In some embodiments, the implanting improves liver antigenic-based defense of the body by performing functions from the group consisting of excretion of at least one complex of IgA, and release of macrophages.

In some embodiments, the improved excretion of at least one complex of IgA, improves body defense against pathologic gut bacteria.

In some embodiments, the improved release of macrophages includes release of at least one Kupfer cell.

In some embodiments, the organ comprises a stomach.

In some embodiments, at least a portion of the portion of stomach tissue is selected from the group consisting of a fundus a body an antrum, and a pylorus.

In some embodiments, the stretching affects at least one bariatric receptor.

In some embodiments, the portion comprises a portion of bladder tissue.

In some embodiments, the stretching at least partially stabilizes an instable detrusor muscle; and in some embodiments the stabilizing prevents spontaneous and uninhibited contraction of the detrusor muscle during filling of bladder 1200.

In some embodiments, at least a portion of the anchor, and/or at least a portion of the connector comprise a material selected from the group consisting of nitinol, stainless steel shape memory materials, metals, and polymers.

In some embodiments, at least a portion of the anchor, and/or at least a portion of the connector, comprise a material selected from the group consisting of nitinol, stainless steel shape memory materials, metals, and polymers.

In some embodiments, at least a portion of the anchor, and/or at least a portion of the connector, include properties from the group consisting of ductile, extendible, extensible, flexible, plastic, resilient, rubbery, springy, tempered, flexile, and pliant.

In some embodiments, at least a portion of the anchor, and/or at least a portion of the connector, comprise a material from the group of biocompatible materials consisting of a polymeric material, a synthetic biostable polymer, a natural polymer, and an inorganic material.

In some embodiments, biostable polymer comprises a material from the group consisting of a polyolefin, a polyurethane, a fluorinated polyolefin, a chlorinated polyolefin, a polyamide, an acrylate polymer, an acrylamide polymer, a vinyl polymer, a polyacetal, a polycarbonate, a polyether, aromatic polyester, a polyester ketone, a polysulfone, a silicone rubber, thermoset polymer, and a polyester imide.

In some embodiments, at least a portion of the anchor, and/or at least a portion of the connector, comprise properties selected from the group consisting of smooth, undulating, and elastic.

In some embodiments, at least a portion of the anchor, and/or at least a portion of the connector, are selected from the group consisting of wires, ribbons, filaments, and cables.

In some embodiments, at least a portion of the at least one connector is substantially flat, and has a shape selected from the group consisting of a pear shape, a fusiform shape, a discoid shape, a triangular shape, and an elongate polygon.

In some embodiments, at least a portion of the anchor, and/or at least a portion of the connector, have a substantially circular cross section having a diameter of at least about 0.1 millimeters. Alternatively, the diameter is at least about 0.2, 0.3, or 0.4 millimeters.

In some embodiments, at least a portion of the anchor, and/or at least a portion of the connector, have a substantially circular cross section having a diameter of no more than about 0.4 millimeters. Alternatively, the diameter is no more that about 0.1, 0.2, or 0.3 millimeters.

In some embodiments, at least a portion of the anchor, and/or at least a portion of the connector have a cross section having greater, and lesser measurements, and the greater measurement is at least about 0.1 millimeters. Alternatively, the measurement is at least about 0.2, 0.3, or 0.4 millimeters.

In some embodiments, at least a portion of the anchor, and/or at least a portion of the connector have a cross section having greater and lesser dimensions, and the greater dimension is no more than about 0.4 millimeters. Alternatively, the greater dimension is no more that about 0.1, 0.2, or 0.3 millimeters.

In some embodiments, at least a portion of the anchor, and/or at least a portion of the connector have a cross section having greater, and lesser dimensions, and the lesser dimension is at least about 0.1 millimeters. Alternatively, the lesser dimension is at least about 0.2, 0.3, or0.4 millimeters.

In some embodiments, at least a portion of the anchor, and/or at least a portion of the connector, has a cross section having greater, and lesser cross sectional dimensions, and the lesser dimension is no more than about 0.4 millimeters. Alternatively, the lesser dimension is no more that about 0.1, 0.2, or 0.3 millimeters.

In some embodiments, at least a portion of the anchor, and/or at least a portion of the connector, have a shape selected from the group consisting of substantially triangular, square, rectangular, round, hexagonal, and logarithmic.

In some embodiments, at least a portion of the anchor, and/or at least a portion of the connector, have a length a length of at least about 2.5 centimeters. Alternatively, the length is at least about 3.0, 3.5, 4.0, or 4.5 centimeters.

In some embodiments, at least a portion of the anchor, and/or at least a portion of the connector, have a length of no more than about 6.5 millimeters. Alternatively, the length is no more that about 6.0, 5.5, 5.0, or 4.5 millimeters.

According to the teachings of the present invention, there is also provided a method for reducing pressure in a portion of kidney tissue. The method consists of, providing a pressure-reducing chamber, sealingly enclosing at least a portion of a kidney in the chamber, and reducing pressure in the chamber, thereby reducing tissue pressure in a portion of kidney tissue of the kidney.

In some embodiments, implanting further comprises implanting the pressure-reducing chamber in vivo.

In some embodiments, the at least one kidney portion includes at least one portion selected from the group consisting of a cortex, medulla, ureter, and pelvis.

In some embodiments, the at least one kidney portion comprises a kidney substantially in its entirety.

In some embodiments, at least a portion of the pressure-reducing chamber comprises a material from the group consisting of a polymeric material, a synthetic biostable polymer, a natural polymer, and an inorganic material.

In some embodiments, the biostable polymer comprises a material from the group consisting of a polyolefin, a polyurethane, a fluorinated polyolefin, a chlorinated polyolefin, a polyamide, an acrylate polymer, an acrylamide polymer, a vinyl polymer, a polyacetal, a polycarbonate, a polyether, an aromatic polyester, a polyester ketone, a polysulfone, a silicone rubber, thermoset polymer, and a polyester imide.

### BRIEF DESCRIPTION OF THE DRAWINGS

The present invention providing methods, and devices for limiting gastric expansion so as to affect motility, volume, hunger sensation, and/or nutrient absorption are described by way of example with reference to the accompanying drawings. With specific reference now to the drawings in detail, it is stressed that the particulars shown are by way of example, and for purposes of illustrative discussion of the preferred method of the present invention only, and are presented in the cause of providing what is believed to be the most useful, and readily understood description of the principles, and conceptual aspects of the invention. In this regard, no attempt is made to show structural details of the invention in more detail than is necessary for a fundamental understanding of the invention, the description taken with the drawings making apparent to those skilled in the art how the methods of the invention may be embodied in practice.

FIGS. 1-2 show side and aerial views of a helical spring anchor used in expanding a portion of an organ, in accordance with an embodiment of the present invention;

FIGS. 3-4 show schematic representations of a kidney and a Bowman's capsule, under the influence of the anchor shown in FIG. 1, in accordance with an embodiment of the present invention;

FIG. 5 shows a tool used for implanting the anchor of FIG. 1, in accordance with an embodiment of the present invention;

FIGS. 6A-6F show alternative embodiments of spring anchors, in accordance with the teachings of the present invention;

FIG. 7 shows a kidney in cross section with a spring anchor implanted in the kidney pelvis, and sutures through the capsule and ribs as an offset, in accordance with an embodiment of the present invention;

FIG. 8 shows embodiments of the spring anchor shown in FIGS. 1-2 used in conjunction with connectors, in accordance with an embodiment of the present invention;

FIGS. 9A-9E show embodiments of connectors, in accordance with the teachings of the present invention;

FIG. 10 shows a kidney in a vacuum box, in accordance with an embodiment of the present invention;

FIGS. 11A-11C show embodiments of tissue stretching devices deployed in stomachs, in cross section, in accordance with embodiments of the present invention;

FIGS. 12A-12B show embodiments of tissue stretching devices deployed in bladders, in cross section, in accordance with embodiments of the present invention;

FIG. 13 shows an embodiment of a tissue-stretching device deployed on a liver, in accordance with an embodiment of the present invention;

FIG. 14 shows a rat stomach interior having been fitted with gastric springs, in accordance with embodiments of the present invention;

FIG. 15 shows another view of the stomach interior of FIG. 14, in accordance with embodiments of the present invention;

FIG. 16 shows the bottom portion of a vacuum chamber inserted into a rat abdomen under the left kidney, in accordance with embodiments of the present invention;

FIG. 17 the vacuum chamber of FIG. 16 with a cover in place, in accordance with embodiments of the present invention; and

FIG. 18 shows the apparatus of FIG. 17 hooked to a gauge demonstrating a reduction in chamber pressure, in accordance with embodiments of the present invention.

### DESCRIPTION OF THE PREFERRED EMBODIMENTS

In broad terms, the present invention relates to methods, and devices for expanding organ tissue so as to reduce interstitial hydrostatic pressure, thereby enhancing organ function.

The principles, and uses of the teachings of the present invention may be better understood with reference to the accompanying description, Figures, and examples. In the Figures, like reference numerals refer to like parts throughout.

Before explaining at least one embodiment of the invention in detail, it is to be understood that the invention is not limited in its application to the details set forth herein. The invention can be implemented with other embodiments, and can be practiced or carried out in various ways. It is also understood that the phraseology, and terminology employed herein is for descriptive purpose, and should not be regarded as limiting.

Generally, the nomenclature used herein, and the laboratory procedures utilized in the present invention include techniques from the fields of biology, engineering, material sciences, medicine, and physics. Such techniques are thoroughly explained in the literature.

Unless otherwise defined, all technical, and scientific terms used herein have the same meaning as commonly understood by one of ordinary skill in the art to which the invention belongs. In addition, the descriptions, materials, methods, and examples are illustrative only, and not intended to be limiting. Methods, and materials similar or equivalent to those described herein can be used in the practice or testing of the present invention.

As used herein, the terms “comprising”, and “including” or grammatical variants thereof are to be taken as specifying the stated features, integers, steps or components but do not preclude the addition of one or more additional features, integers, steps, components or groups thereof. This term encompasses the terms “consisting of”, and “consisting essentially of”.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic, and novel characteristics of the claimed composition, device or method.

As used herein, “a” or “an” mean “at least one” or “one or more”. The use of the phrase “one or more” herein does not alter this intended meaning of “a” or an

The term “method” refers to manners, means, techniques, and procedures for accomplishing a given task including, but not limited to, those manners, means, techniques, and procedures either known to, or readily developed from known manners, means, techniques, and procedures by practitioners of the chemical, pharmacological, biological, biochemical, and medical arts. Implementation of the methods of the present invention involves performing or completing selected tasks or steps manually, automatically, or a combination thereof.

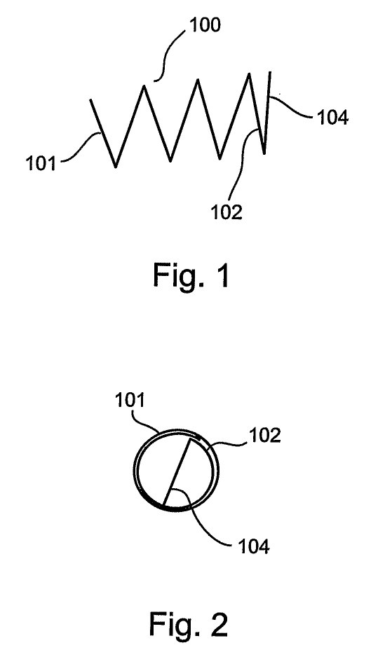

FIGS. 1 and 2 show side and aerial views respectively of a helical spring anchor 100 that is compressible by pressing a spring first end 101 toward a spring second end 102.

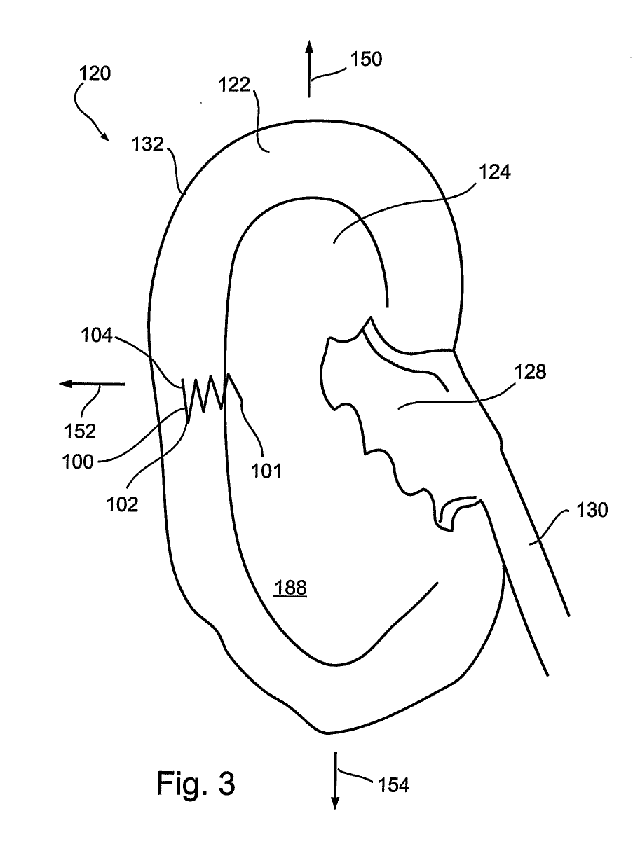

In FIG. 3, spring anchor 100 is shown implanted in a cortex 122 of a kidney. Spring 100 is compressed prior to implantation in cortex 122. Upon introduction into cortex 122, spring 100 is released so that ends 101 and 102 move away from each other, for example end 102 moving in a direction 152, and end 101 moving in an opposite direction. The force applied by spring 100 causes a kidney capsule 132 to expand substantially radially outward in direction 152. When placed in a portion of an organ, for example kidney tissue 188, (FIG. 3) stretch of tissue 188 causes a reduction in pressure that enhances function of an organ that will be described below and demonstrated in “Experimental Results”.

Referring back to FIGS. 1 and 2, in embodiments, spring 100 is placed perpendicular, parallel or at any angle therebetween with respect to capsule 132, thereby stretching a kidney tissue 188 and thereby improving organ function. In embodiments (not shown), multiple springs 100 are expanded in a kidney 120 at multiple locations, causing capsule 132 to expand radially outward, and stretching kidney tissue 188, for example located in a kidney cortex 122, a kidney medullar 124, a kidney pelvis 128 (below) or, a kidney ureter 130.

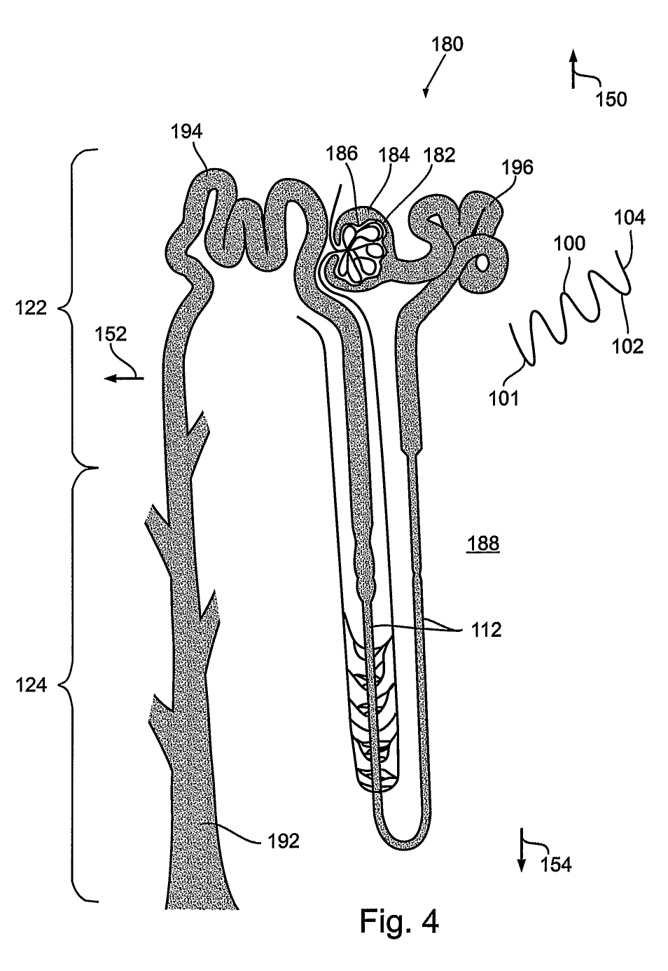

FIG. 4 shows a typical nephron 180 having glomerular capillaries 182, separated from a renal corpuscle 184 by a Bowman's space 186. As spring 100 expands, ends 101 and 102 move away from each other so that a portion of tissue 188 adjacent to nephron 180 stretches. Stretched tissue 188 thereby expands corpuscle 184, increasing the volume of, and reducing pressure within Bowman's space 186. The reduced pressure in Bowman's space typically causes a higher filtration rate between capillaries 182 and corpuscle 184.

Additionally, it is postulated that the stretch in tissue 188 may cause reduction in interstitial pressure in loop of Henle 112, a distal convoluted tube 194, a proximal convoluted tube 196, and/or a collecting duct 192, thereby enhancing the filtration rate associated with each of these structures.

The enhancement of Glomerular Filtration Rate (GFR) is governed by the formula as presented in the text Physiology by Berne and Levy:

GFR=K_{f}[(P_{GC}−P_{BS})−(Π_{GC}−Π_{BS})]

Wherein the following nomenclature is used:

i) Kf: the Ultra Filtration Constant

ii) PGC: Hydrostatic Pressure of Glomerular Capillary

iii) PBS: Hydrostatic Pressure of Bowman's Space

iv) ΠGC: osmotic pressure of Glomerular Capillary

v) ΠBS: osmotic pressure of Bowman's Space

GFR=K_{f}[(44_{[mmHg]}−(12_{[mmHg]}))−(−34_{[mmHg]}−0)]

As noted in the equation above, a P_{BS} reduction by 10 [mmHg] causes a GFR elevation of 11%.

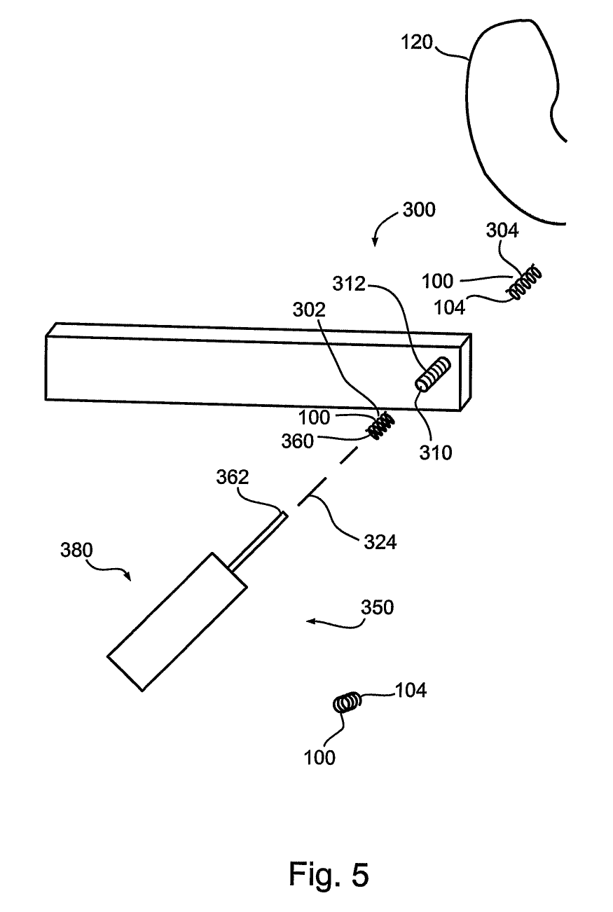

FIG. 5, shows a typical instrument 300 used for insertion of spring 100 into kidney 120. Spring 100 is pushed into a passage 310. A driver 380 is pushed along an axis 324 leading into passage 310, and prongs 362 of driver 380 are placed around a spring abutment 104. Driver 380 is rotated so that spring 100 follows a rifling 312, and forms a compressed configuration 302 as spring 100 compresses against a portion of kidney 120. As driver 380 is further rotated, spring 100 is driven into kidney in compressed configuration 302, and, in the softer tissue of kidney 120 expands into an expanded configuration 304, thereby stretching a portion of interstitial tissue of kidney 120 (FIG. 3).

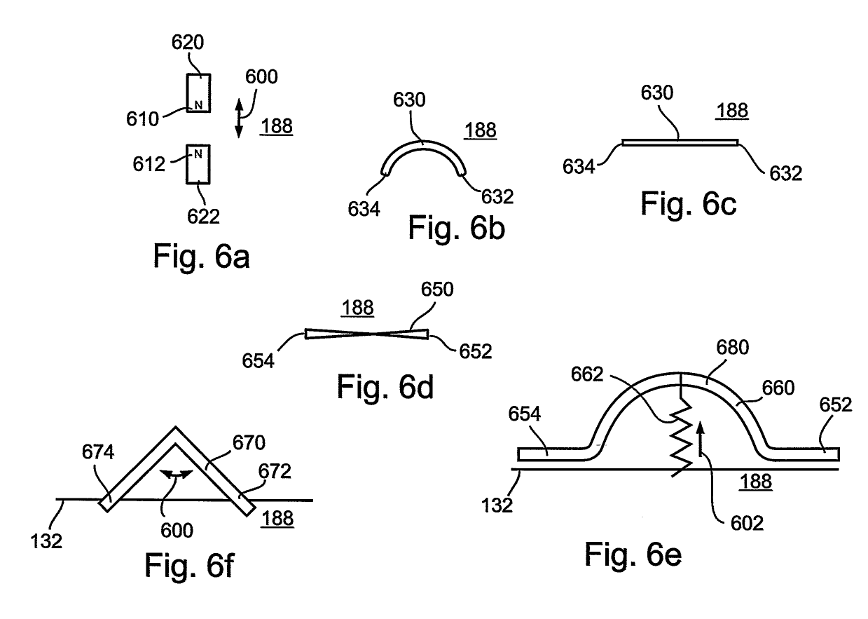

Spring 100 (FIG. 2) is but one of the many devices that can be used in stretching kidney tissue 188. FIG. 6A shows a first magnet 620 and a second magnet 622 in which same polarities 610 and 612 are aligned and facing toward each other. A repulsive force 600 is thereby created, pushing first magnet 620 away from second magnet 622 so that when implanted in a portion of tissue 188, tissue 188 is stretched.

FIG. 6B shows a leaf spring 630 that has been bent to bring an end 632 toward a second end 634. Bent spring 630 is implanted in tissue 188 and released as seen in FIG. 6C. As spring 630 straightens, ends 632 and 634 stretch tissue 188.

In an alternative embodiment for stretching tissue 188, FIG. 6D shows a rigid anchor 650 having a first end 652, and a second end 654. Initially, tissue 188 is stretched, after which rigid anchor 650 is implanted in tissue 188 to maintain tissue 188 in the stretched state.

FIG. 6E shows an offset frame 660 that is substantially rigid, having first end 652, and second end 654 projecting from either side of an offset bow 680. A tensioned spring 662 spans from bow 680 to tissue 188, and pulls capsule 132 in a direction 602, thereby stretching tissue 188. Using offset bow 680, any biocompatible elastomeric band or device is optionally used in place of tensioned spring 662, as is easily understood by those familiar with the art.

FIG. 6F shows a leaf spring 670 bent at right angle with arms 672 and 674 implanted into tissue 188, just below kidney capsule 132. As spring 670 is released, arms 672 and 674 stretch capsule 132 in directions 600, thereby stretching tissue 188.

In an alternative embodiment, arms 672, and 674 are attached to capsule 132 using biological glue, for example carboxymethyl cellulose.

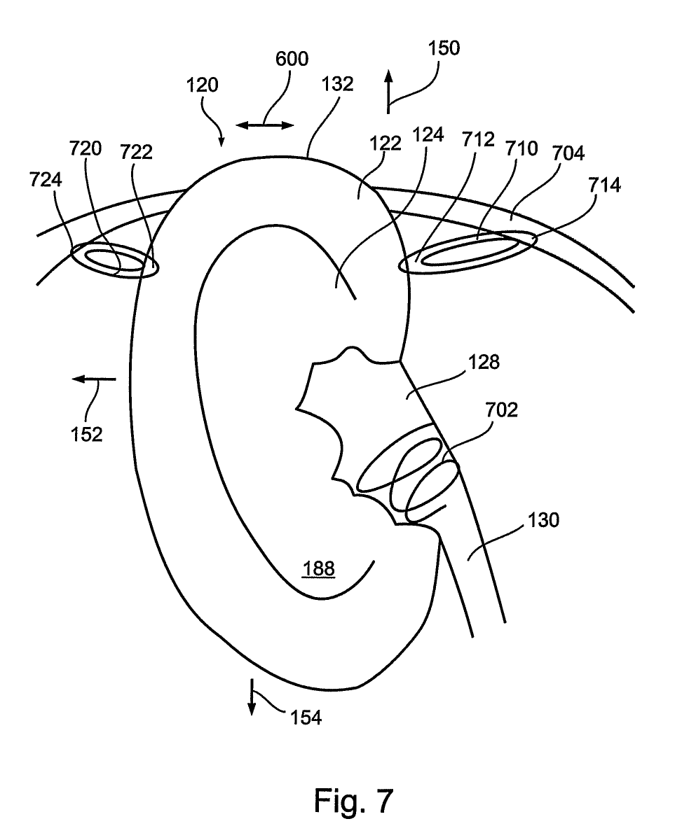

FIG. 7 shows a coiled spring 702 that has been expanded inside kidney pelvis 128. It is postulated that such expansion will also favorably affect hydrostatic pressure within corpuscle 184 (FIG. 4).

In an alternative embodiment, a first suture loop 710, and a second suture loop 720 have been attached to kidney capsule 132 with proximal loops 712, and 722 respectively. Distal loops 714 and 724 have been anchored to a rib 704 that acts as an offset. The generated tension pulls kidney 120 in directions 600, thereby stretching tissue 188. While rib 704 is depicted as being used as an offset, in embodiments other body organs and/or tissue are used as an offset, for example parts of the vertebral column.

Additionally, alternatives to anchor loops 710, and 712 may be contemplated, as will be easily appreciated by those familiar with the art, including, inter alia: Different spring shapes (FIGS. 6A-6F), or varying materials to influence resilience.

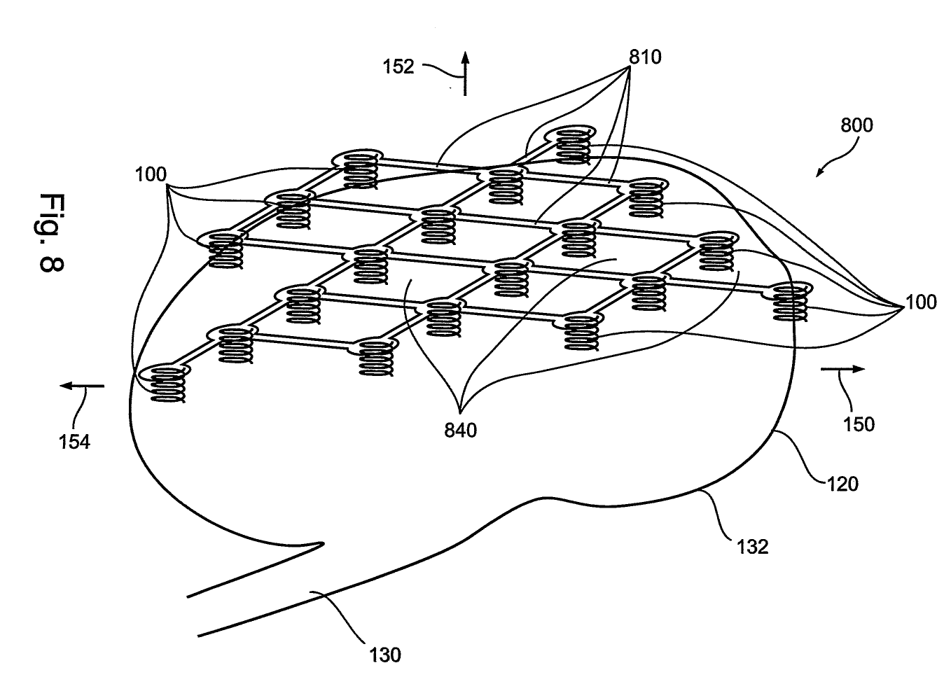

FIG. 8 shows spring anchors 100 implanted in kidney 120, and connected to a series of connectors 810 that have been assembled into a grid 800. In an exemplary embodiment, grid 800 is contoured to the shape of the adjacent tissue of kidney 120 so that springs 100 pull kidney capsule radially outward in direction 150, 152, and/or 154, depending upon placement.

While connector grid 800 is shown as having square spaces 840 between connectors 810, a variety of configurations are possible. For example, grid 800 may comprise triangle shaped spaces or even comprise a substantially rigid mesh or net.

While connector grid 800 is shown as a single unit, in embodiments grid 800 comprises multiple separate connectors 810 that are joined to form grid 800, separate connectors 810 joined, for example, at anchors 100. Alternatively, separate multiple connectors 810 are fashioned into a variety of configurations, for example two connectors 810 forming linear or non linear patterns; and multiple connectors forming open or closed polygonal shapes.

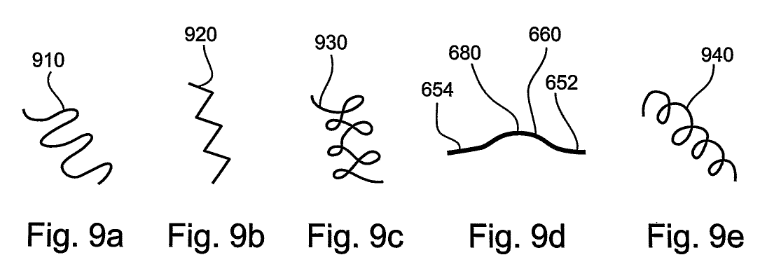

FIGS. 9A-9E demonstrate embodiments of connectors 810 that can be used in forming grid 800 either as a single unit or made up of multiple units. Connector 910 is optionally configured with any one of a variety of shapes, including: an undulate shape connector 910, a zigzag shaped connector 920, a small looped connector 930, and a large looped connector 940.

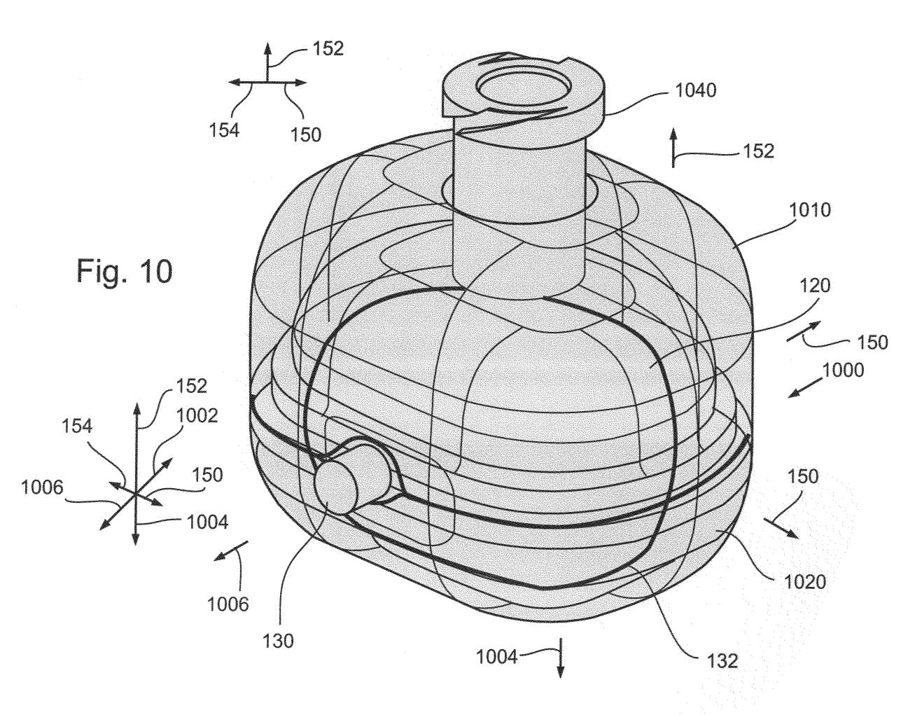

FIG. 10 is a vacuum box 1000, having a top 1010, and a bottom 1020 enclosing kidney 120 and allowing kidney ureter 130 to pass out of vacuum box 1000. In an exemplary embodiment, and as described in “Experimental Results”, below, pressure in box 1000 is reduced below atmospheric pressure by withdrawing air via a vacuum passage 1040. Reduction of pressure in box 1000 causes expansion of kidney capsule 132, thereby stretching kidney 120 in directions 150, 152, and/or 154. Additionally, because box totally surrounds kidney 120, expansion in directions 1002, 1004, and 1006 occur, so that expansion of kidney 120 is in three dimensions.

While vacuum box 1000 is shown totally surrounding kidney 120, there are many configurations in which box 1000 optionally affects a smaller portion of kidney 120 and, for example, seals against kidney capsule 132, thereby providing reduced pressure to tissue associated with the portion of kidney 120.

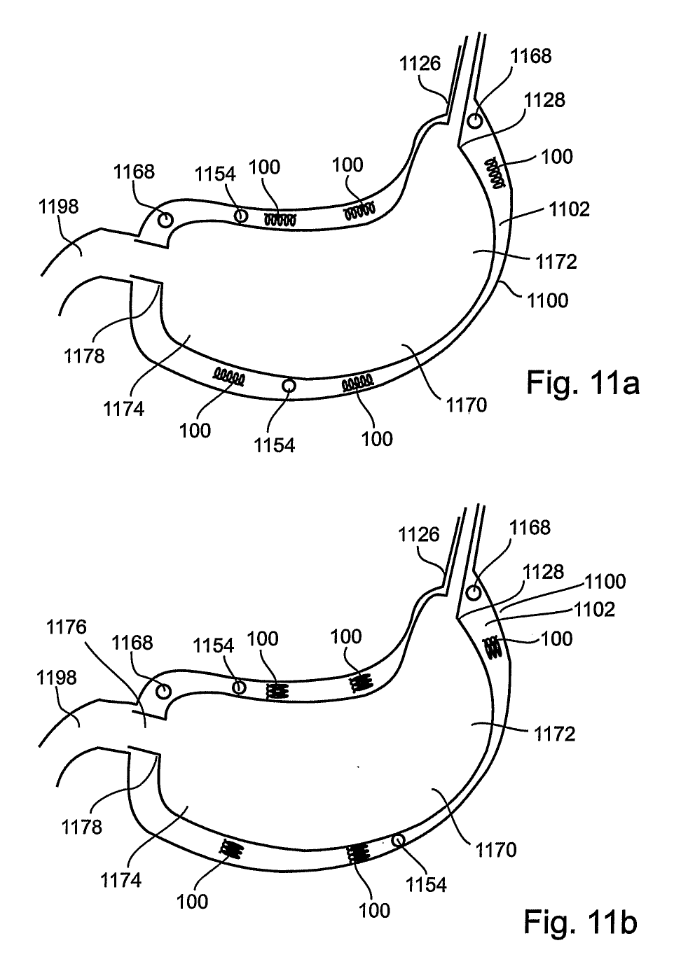

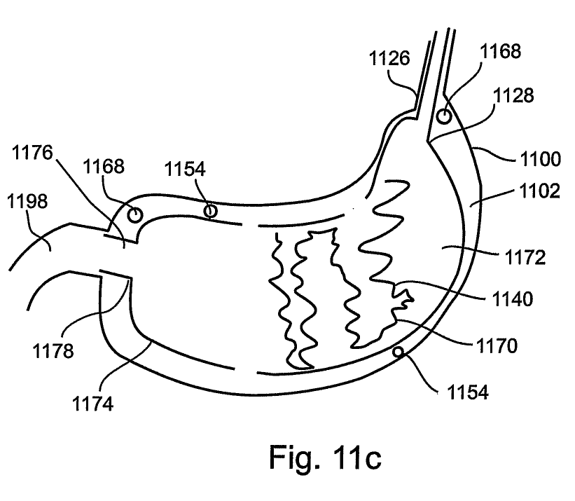

FIGS. 11A-11B show stomachs 1100 in cross section with springs 100 that are placed in a gastric wall 1102 in the compressed state. When springs 100 are allowed to expand, in a gastric wall 1102, as demonstrated in “Experimental Results”, below, gastric wall 1102 stretches thereby affecting intraganglionic laminar endings (IGLEs) 1154 noted above.

By stretching IGLEs 1154, it is postulated that the recipient of springs 100 will feel satiated even though a full meal has not been ingested.

In FIG. 11A, springs 100 are placed parallel to gastric wall 1102, and in FIG. 11B, springs 100 are placed perpendicular to gastric wall 1102, both configurations and all angles therebetween being postulated to affect IGLEs 1154 in the above-noted manner.

The present invention contemplates application of springs 100 to a variety of gastric-related tissue 1102. For example, springs 100 are optionally implanted in tissue having high density IGLEs 1154, for example in an esophagus 1126, a fundus 1172, an antrum 1170, a gastric body 1174, and/or a pylorus 1176.

Alternatively, springs 100 may be used to stretch tissue intramuscular arrays (IMAs) 1168 that are known to be more numerous in an esophageal sphincter 1128, and a pyloric sphincter 1178.

FIG. 11C shows a mesh spring 1140 that has been expanded inside stomach 1100 to stretch stomach wall 1102 thereby affecting receptors including IGLEs 1154, and IMAs 1168.

As with springs 100, the position of mesh 1140 may be throughout all gastric tissue 1102 or placed in individual areas of gastric tissue 1102, for example in esophagus 1126, fundus 1172, antrum 1170, gastric body 1174, and/or pylorus 1176.

The exact mechanisms of providing satiety and fullness sensations to an obese individual are not fully known to the bariatric community. It is believed that restricting volume of stomach 1100 causes receptors 1154 and 1168 to register satiation, and/or fullness, thereby favorably influencing diet, and aiding in weight loss. Any reference to receptors 1154, and 1168 a priori refers to any gastric receptors presently identified, and those that will be identified, for example by bariatric researchers, in the future.

Additionally, the methods, and/or configuration of material applied to stomach 1100, for example size, and/or placement of springs 100, and/or connectors 810 (FIG. 8), a priori include any modifications that are discovered to be efficacious or become known in the future.

As used herein gastric tissue 1102 refers to any portion of gastric-related tissue 1102 that is part of, or near, stomach 1100, for example, inter alia, esophagus 1126, fundus 1172, antrum 1170, body 1174, pylorus 1176, pyloric sphincter 1178, and/or an intestine 1198.

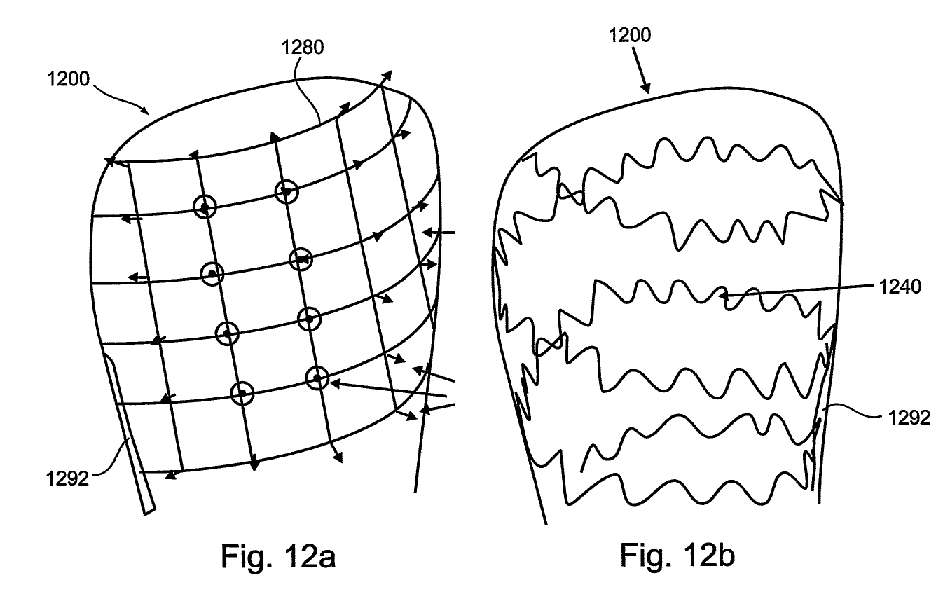

FIG. 12A shows a bladder 1200 fitted with a grid 1280 that comprises an embodiment of tissue stretching grid 800 shown in FIG. 8. Optionally, grid 1280 is attached to bladder 1200 using a suitable pharmaceutically acceptable adhesive, for example carboxymethyl cellulose, thereby aiding in controlling function of bladder 1200, as explained below.

FIG. 12B shows bladder 1200 fitted with a mesh spring 1240 that comprises an embodiment of mesh spring 1140 shown in FIG. 11C.

It is postulated that embodiments of grid 1280, and mesh spring 1240 will have particular use in treating instability of a detrusor muscle 1292 by preventing spontaneous and uninhibited contraction of detrusor muscle 1292 during filling of bladder 1200.

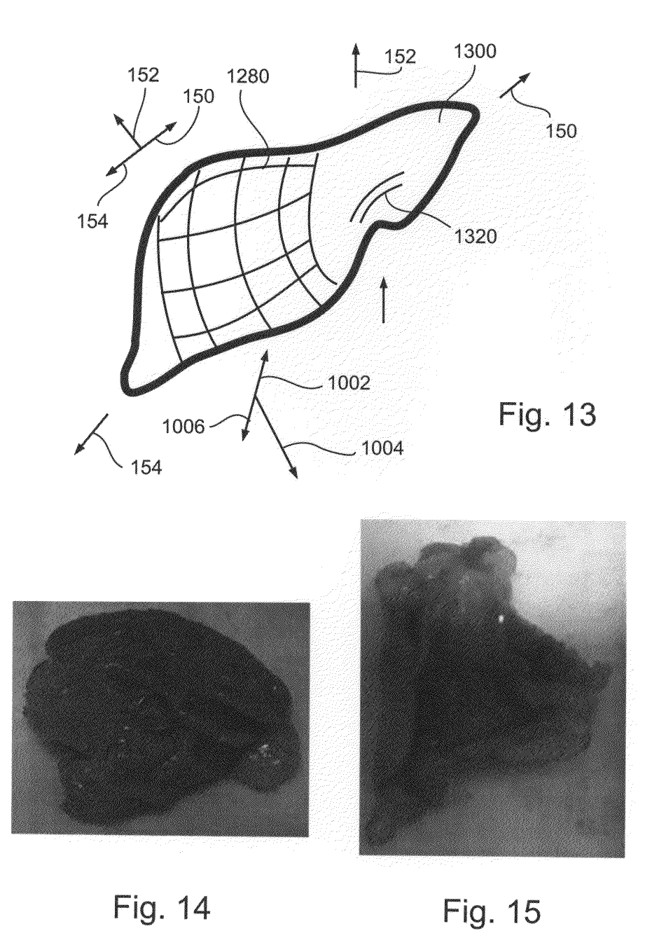

FIG. 13 shows a liver 1300 fitted with tissue stretching device 1280 that is optionally attached to liver 1300 using a suitable pharmaceutically acceptable adhesive. It is postulated that by stretching liver 1300 in at least one of directions 150, 152, 154, 1002, 1004, and 1006, the resultant increased liver volume will result in greater blood flow volume through a hepatic blood vessel 1320. It is postulated that the increased blood flow will help alleviate ascites, and foster better liver function.

The better liver function optionally is evident through improvement of at least one liver function, including, inter alia:

Increasing homeostatic compounds consisting of glucose, proteins, fat, cholesterol, hormones, and vitamins; Increasing homeostasis of a vitamin from the group consisting of: vitamins A, D, E, and K; Improving liver synthesis of at least one compound from the group consisting of: proteins, bile acids, cholesterol and at least one clotting factor; Improving liver storage of at least one compound from the group consisting of: vitamins, and cholesterol; Improving liver excretion of at least one compound from the group consisting of: cholesterol, bile acids, phospholipids, bilirubin, drugs, and poisons; Improving liver filtration of at least one compound from the group consisting of: gut poisons, nutrients, sugar, fat, bilirubin, bile acids, and immunoglobulins; Improving filtration of nutrients includes filtration of at least one compound from the group consisting of: amino acids, immunoglobulins including IgA; Improving antigenic-based defense of the body by improving functions from the group consisting of: excretion of at least one complex of IgA, and release of macrophages.

### EXPERIMENTAL RESULTS

### Example 1

### Effects of Kidney Spring Implantation

To investigate the effects of implantation of kidney springs such as kidney springs 100 of the present invention (FIG. 1) on various indicators of kidney function, the following implantation and examination procedures were performed.

### Kidney Spring Implantation Procedure

A Sprague-Dawley (SD) rat, weighing about 250 grams, was anesthetized. A laparotomy was performed and the left kidney was exposed.

A length of surgical grade nitinol wire having a diameter of 0.25 millimeter was coiled to make helical springs, each spring having a helical diameter of 3 millimeter, a length of about 4 millimeters and 4 turns.

Two such helical springs were screwed into the rat left kidney using a specially designed screwdriver and delivery device, as seen in FIG. 5, and as described above. The right rat kidney served as a control. The laparotomies were closed and the rat was revived.

### Kidney Examination Procedure at Ten Days

Ten days after spring implantation, the rat was subjected to a second laparotomy procedure to allow macroscopic visualization of hepatic integrity and to check for the presence of bleeding that would indicate trauma caused by the springs.

Additionally, inulin and saline were infused for the purpose of establishing Glomerular Filtration Rate (GFR). Inulin is an inert polysaccharide, polyfructosan, [C₆H₁₀O₅] which readily passes through the glomeruli into the urine without being reabsorbed by the renal tubules. Inulin clearance is an excellent indicator of GFR.

The inulin clearance test was performed by injecting inulin into the bloodstream, waiting for it to be distributed, and then measuring plasma inulin and urine inulin concentrations.

To collect urine samples from each kidney independently, the left kidney ureter was incised from its attachment to the urinary bladder and urine was collected through a catheter attached through the left ureter. The right kidney ureter remained intact and urine was collected through a catheter attached to the urinary bladder.

Urine samples were taken at 30-minute intervals following inulin injection, over a period of 2 hours, from the left ureter (U1, U2, U3 and U4) and from the urinary bladder (U1N, U2N, U3N and U4N). Inulin levels (Inulin OD), of each sample were measured. Also measured was the volume of urine (VU) in □1.

Based on the urine measurements, urine flow rate [ml/min] (Vf); urine inulin concentration in mg/100 ml (UIn); and inulin amount in milligrams (UIn*dil) were calculated.

Urine analysis results are presented in Tables 1 and 2 below.

Samples of blood were removed from the Jugular vein at intervals of 30 minutes over a period of 90 minutes (B1, B2, and B3) and tested for sodium (Na) and potassium (K) concentrations, in mEq/L, in order to establish that the rat did not undergo dehydration. Inulin levels (Inulin OD) were measured and the plasma inulin concentration (PIn), in mg/100 ml, and plasma inulin amount in milligrams (PIn*dil) were calculated.

Blood test results are presented in Table 3 below.

GFR was calculated according to the formula:

GFR=[(UIn*dil)×Vf]/(PIn*dil).

### Results

The kidneys appeared normal macroscopically and all springs were in place.

There was no evidence of blood during macroscopic examination of the kidneys. The urinary bladder was lucent and without blood.

As shown in Table 1 and Table 2, the implanted kidney displayed an increase in GFR of approximately 15% over the control kidney.

TABLE 1
Left (implanted) Rat Kidney Function

| | | Time | | Inulin | | | |
|---|---|---|---|---|---|---|---|
| | VU | min | Vf | OD | UIn | UIn*dil | GFR |
| U1 | 0.23 | 30 | 0.0077 | 564 | 263.7609 | 10550.437 | 2.7242 |
| U2 | 0.236 | 30 | 0.0079 | 574 | 268.4375 | 10737.502 | 2.5337 |
| U3 | 0.232 | 30 | 0.0077 | 450 | 210.4476 | 8417.902 | 2.3274 |
| U4 | 0.25 | 30 | 0.0083 | 438 | 204.8356 | 8193.425 | 2.3175 |

TABLE 2
Right (control) Rat Kidney Function

| | | Time | | Inulin | | | |
|---|---|---|---|---|---|---|---|
| | VU | min | Vf | OD | UIn | UIn*dil | GFR |
| U1N | 0.28 | 30 | 0.0093 | 613 | 286.6763 | 11467.053 | 2.1964 |
| U2N | 0.293 | 30 | 0.0098 | 548 | 256.2784 | 10251.134 | 2.3389 |
| U3N | 0.286 | 30 | 0.0095 | 444 | 207.6416 | 8305.663 | 2.0156 |
| U0N | 0.318 | 30 | 0.0106 | 458 | 214.1888 | 8567.554 | 2.0493 |

TABLE 3
Blood sample analysis

| | | | Inulin | | |
|---|---|---|---|---|---|
| | Na | K | OD | BIn | BIn*dil |
| B1 | 144.5 | 3.21 | 77 | 36.01 | 36.010 |
| B2 | 145.7 | 3.4 | 84 | 39.284 | 39.284 |
| B3 | 146.2 | 3.49 | 84 | 39.284 | 39.284 |

### Example 2

### Effects of Stomach Springs Implantation

To study the effects of implantation of kidney springs such as kidney springs 100 of the present invention (FIG. 1), the following Implantation and Examination procedures were performed:

### Stomach Spring Implantation Procedure

Four Sprague-Dawley (SD) rats, each weighing about 250 grams, were anesthetized using using ketamin/xylasine.

A laparotomy was performed on each rat, exposing the stomach.

Five helical spiral springs of surgical grade nitinol, as described in detail in Example 1, were screwed to the anterior aspect of each stomach body.

Two rats (rats 3 and 4) had the springs immediately removed and were observed for bleeding. All rats were then surgically closed.

Two months later one rat (rat 2) was sacrificed and springs were examined macroscopically for corrosion.

### Results

No significant bleeding or significant damage occurred in rats 3 and 4 following immediate removal of the springs.

There was no macroscopic evidence of corrosion present on the springs from rat 2 at two months. Additionally, there was no evidence of bleeding in rat 2.

It should be noted that since all rats survived throughout the experiment, it is believed no rat experienced significant bleeding.

FIGS. 14-15 show inside aspects of the stomach of one rat (rat 2), two months after having been fitted with gastric springs, and showing appropriate organ integrity.

### Example 3

### Establishing Kidney Vacuum Chamber Efficacy

To evaluate the feasibility of enclosing a kidney in a chamber and subjecting the kidney to a partial vacuum, the following Implantation and Examination procedures were performed:

### Vaccum Chamber Procedure

One SD rat, weighing 453 grams, was anesthetized using ketamin/xylasine.

A laparotomy was preformed on the rat, exposing the left kidney.

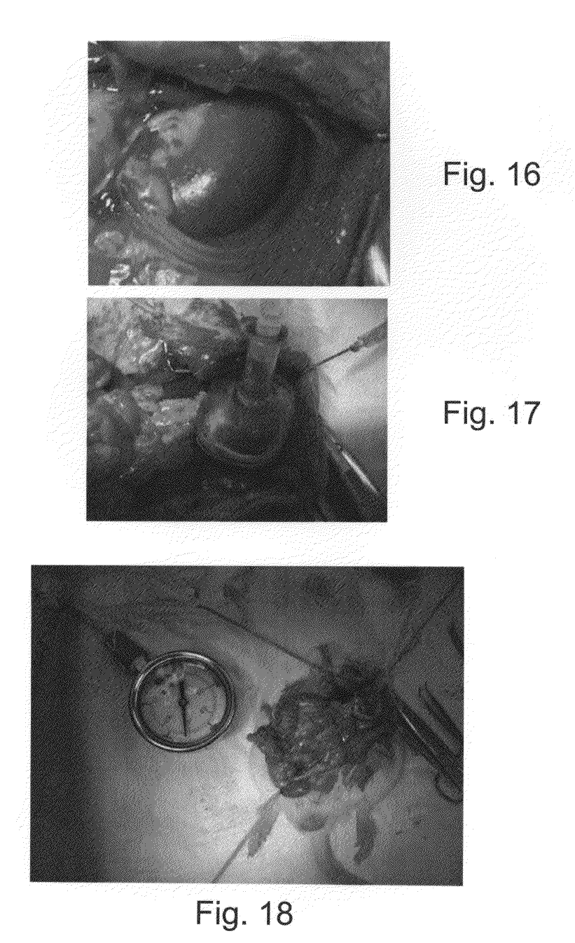

As seen in FIG. 16, a bottom portion of a vacuum chamber was inserted into the rat abdomen under the left kidney. The vacuum chamber was then closed by addition of an upper portion.

FIG. 17 shows the left kidney inside the closed vacuum chamber of FIG. 16, following which the chamber was sealed with silicone. The right kidney served as a control.

A vacuum pump was attached to the chamber and, as seen in FIG. 18, the reduction in pressure within the chamber was measured.

### Kidney Examination Procedure

To determine the efficacy of the vacuum in improving kidney function, the vacuum is maintained in the chamber to continue reduced pressure forces on the kidney.

Following a period of time, for example two hours, the rat is opened to allow macroscopic visualization of hepatic integrity.

In order to assess kidney function, inulin is injected for the purpose of establishing GFR. Urine samples are collected from each kidney independently, by incising the left kidney ureter from its attachment to the urinary bladder and collecting urine through a catheter attached through the left ureter. The right kidney ureter remains intact and urine is collected through a catheter attached to the urinary bladder.

Urine samples are taken at 30-minute intervals following inulin injection, over a period of 2 hours, from the left ureter and from the urinary bladder. Inulin levels of each sample and volume of urine are measured. Based on the urine measurements, urine flow rate; urine inulin concentration; and inulin amount in milligrams are calculated.

Samples of blood are removed from the Jugular vein at intervals of 30 minutes over a period of 90 minutes and tested for sodium and potassium concentrations, in order to establish that the rat does not undergo dehydration. Inulin levels are measured and the plasma inulin concentration and plasma amount are calculated. GFR is calculated as described hereinabove.

It is expected that during the life of this patent many relevant delivery systems will be developed, and the scope of the various embodiments of the invention, and the various methods of implementation are intended to include all such new technologies a priori.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications, and variations will be apparent to those skilled in the art. Accordingly, it is intended to embrace all such alternatives, modifications, and variations that fall within the spirit, and broad scope of the appended claims. All publications, patents, and patent applications mentioned in this specification are herein incorporated in their entirety by reference to the specification, to the same extent as if each individual publication, patent or patent application was specifically, and individually indicated to be incorporated herein by reference. In addition, citation or identification of any reference in this application shall not be construed as an admission that such reference is available as prior art to the present invention.

1-24. (canceled)

25. A method of modifying a physiological behavior of an organ, comprising:
(a) stretching at least a portion of said organ; and
(b) maintaining said stretching for a period of time sufficient to affect a physiological behavior of the organ.
(c) Where the organs are selected form a group consisting of a kidney, a stomach, a liver and a urinary bladder.

26. A method according to claim 25, wherein said stretching comprises reducing an internal pressure in at least said portion.

27. A method according to claim 25, wherein said stretching comprises reducing an internal pressure in substantially all of said organ.

28. A method according to claim 25, wherein stretching comprises stretching an outer layer of said organ.

29. A method according to claim 25, wherein stretching comprises applying negative pressure to said portion.

30. A method according to claim 25, wherein applying negative pressure to said portion comprising surrounding at least said portion with a chamber.

31. A method according to claim 25, wherein stretching comprises implanting a spring in said portion.

32. A method according to claim 31, wherein said spring is implanted along an outer layer of said organ.

33. A method according to claim 31, wherein said spring is implanted perpendicular to an outer layer of said organ.

34. A method according to claim 31, wherein said spring applies force along an outer layer of said organ.

35. A method according to claim 31, wherein said spring applies force perpendicular to an outer layer of said organ.

36. A method according to claim 31, wherein said implanting comprises implanting said spring in a compressed configuration thereof.

37. A method according to claim 25, wherein stretching comprises anchoring said portion to tissue outside said organ.

38. A method according to claim 25, wherein said organ comprises a kidney and wherein said stretching causes an increase in Glomerular Filtration Rate (GFR).

39. A method according to claim 25, wherein said stretching comprises reducing an internal pressure of said volume by at least 10 mmHg.

40. A method according to claim 25, wherein said organ comprises a stomach and wherein said stretching causes a feeling of satiety.

41. A method according to claim 25, wherein said organ comprises a liver and wherein said stretching causes an improvement in liver function.

42. A method according to claim 25, wherein said organ comprises a urinary bladder and wherein said stretching reduces undesirable contraction of the bladder.

43. An elastic medical implant configured to fit completely in a wall of a stomach and stretch said wall, without perforating said wall.

44. An elastic medical implant configured to fit completely in a cortex of a kidney and stretch said cortex without perforation thereof.

45. A sheet comprising a plurality of elastic elements arranged in a direction perpendicular to said sheet and configured for attachment to an in-vivo-organ.

46. A vacuum chamber, comprising:
(a) a housing defining a volume and adapted for implantation in a body;
(b) a port defined in said volume and adapted for attachment to a source of vacuum; and
(c) at least one port defined by said housing and adapted for passage of body tissue including at least one lumen therethrough and adapted to seal against said tissue without blocking said lumen.

47. A chamber according to claim 46, adapted to fit a kidney.

48. A chamber according to claim 46, comprising a source of vacuum adapted to maintain a vacuum level in said chamber for a period of time greater than 2 hours.
